(19) United States
(12) Patent Application Publication
(10) Pub. No.: US 2026/0250767 A1
(43) Pub. Date: Aug. 27, 2026

(54) Treatment Of Inflammatory Bowel Disease In Subjects Having Loss-Of-Function (LOF) Gene Variants

(71) Applicant: Regeneron Pharmaceuticals, Inc., Tarrytown, NY (US)

(72) Inventors: Anna Alkelai, Tarrytown, NY (US); Alan Shuldiner, Tarrytown, NY (US); Joshua Backman, Tarrytown, NY (US); Antoine Baldassari, Tarrytown, NY (US)

(21) Appl. No.: 19/535,173

(22) Filed: Feb. 10, 2026

Related U.S. Application Data
(60) Provisional application No. 63/757,194, filed on Feb. 11, 2025.

(51) Int. Cl. C12Q 1/6883; C12Q 1/26

(52) U.S. Cl. CPC C12Q 1/6883; C12Q 1/26; C12Q 2600/106; C12Q 2600/156; G01N 2333/90241

(57) ABSTRACT

The present disclosure generally relates to the treatment of subjects having IBD or at risk of developing IBD by administering an Indoleamine 2,3-Dioxygenase 2 (IDO2) agonist or a SMAD Family Member 2 (SMAD2) agonist to the subject.

### FIELD

The present disclosure generally relates to the treatment of subjects having IBD or at risk of developing IBD, by administering an Indoleamine 2,3-Dioxygenase 2 (IDO2) agonist or a SMAD Family Member 2 (SMAD2) agonist to the subject, and to methods of identifying subjects having an increased risk of developing IBD.

### BACKGROUND

Inflammatory bowel disease (IBD) is a genetically heterogeneous, chronic inflammatory disorder of the colon and small intestine initiated by the inappropriate immune response to commensal microbiota in the gastrointestinal tract and with an average age of onset at 30 years. IBD causes over 50,000 deaths annually. Severe, monogenic forms of IBD can present with pediatric age of onset (<18 years) and have been attributed to rare, highly-penetrant variants in about 50 Mendelian genes. However, the genetic architecture of early onset inflammatory bowel disease (EO-IBD) is poorly understood, and the majority of patients remain genetically undiagnosed. The causes of IBD are complex, and contributing factors may include diet, genetics, and the composition of an individual's gut microflora.

Indoleamine 2,3-Dioxygenase 2 (IDO2) is a gene that is 81,779 bases long and located at 8p11.21. IDO2 encodes an enzyme that play a key role in metabolizing tryptophan in the kynurenine pathway. The protein encoded by this gene is 407 amino acids long and 45424 Da. IDO2 may play a role as a negative regulator of IDO1 by competing for heme-binding with IDO1. IDO2 is expressed in human tumors in an inactive form: tryptophan degradation is entirely provided by IDO1 in these cells.

SMAD Family Member 2 (SMAD2) is a gene that is 122,190 bases long and located at 18q21.1. The protein encoded by this gene is 467 amino acids long and 52306 Da. SMAD are a group of related intracellular proteins critical for transmitting to the nucleus signals from the transforming growth factor-β (TGFβ) superfamily at the cell surface. SMAD proteins mediate TGF-beta signaling to regulate cell growth and differentiation. SMAD protein mediates their effects through protein-protein and protein-DNA interactions. SMAD2 play an essential role in mediating TGFβ-superfamily signals in development and disease. Diseases associated with SMAD2 include Loeys-Dietz syndrome and congenital heart defects, multiple types, with or without heterotaxy.

### SUMMARY

The present disclosure provides methods of treating a subject having inflammatory bowel disease, or at risk of developing inflammatory bowel disease, the methods comprising administering an IDO2 agonist to the subject.

The present disclosure also provides methods of treating a subject having inflammatory bowel disease or at risk of developing inflammatory bowel disease by administering an IBD therapeutic agent, the methods comprising: determining or having determined whether the subject has an IDO2 variant nucleic acid molecule, by: obtaining or having obtained a biological sample from the subject; and performing or having performed a sequence analysis on the biological sample to determine if the subject has a genotype comprising an IDO2 variant nucleic acid molecule; and administering or continuing to administer the inflammatory bowel disease therapeutic agent in an amount that is the same as a standard dosage amount to a subject that is IDO2 reference; or administering or continuing to administer the inflammatory bowel disease therapeutic agent in an amount that is the same as or less than a standard dosage amount, and/or administering an IDO2 agonist to a subject that is heterozygous or homozygous for the IDO2 variant nucleic acid molecule; wherein the presence the IDO2 variant nucleic acid molecule indicates the subject has an increased risk of developing inflammatory bowel disease.

The present disclosure also provides methods of identifying a subject having an increased risk of developing inflammatory bowel disease, the methods comprising: determining or having determined the presence or absence of an IDO2 variant nucleic acid molecule in a biological sample obtained from the subject; wherein: when the subject is IDO2 reference, then the subject has a decreased risk of developing inflammatory bowel disease; and when the subject is heterozygous or homozygous for the IDO2 variant nucleic acid molecule, then the subject has an increased risk of developing inflammatory bowel disease.

The present disclosure also provides inflammatory bowel disease therapeutic agents for use in the treatment or prevention of inflammatory bowel disease in a subject having an IDO2 variant nucleic acid molecule.

The present disclosure also provides IDO2 agonists for use in the treatment or prevention of inflammatory bowel disease in a subject that is IDO2 heterozygous or homozygous for an IDO2 variant nucleic acid molecule.

The present disclosure provides methods of treating a subject having inflammatory bowel disease, or at risk of developing inflammatory bowel disease, the methods comprising administering a SMAD2 agonist to the subject.

The present disclosure also provides methods of treating a subject having inflammatory bowel disease or at risk of developing inflammatory bowel disease by administering an IBD therapeutic agent, the methods comprising: determining or having determined whether the subject has a SMAD2 variant nucleic acid molecule, by: obtaining or having obtained a biological sample from the subject; and performing or having performed a sequence analysis on the biological sample to determine if the subject has a genotype comprising a SMAD2 variant nucleic acid molecule; and administering or continuing to administer the inflammatory bowel disease therapeutic agent in an amount that is the same as a standard dosage amount to a subject that is SMAD2 reference; or administering or continuing to administer the inflammatory bowel disease therapeutic agent in an amount that is the same as or less than a standard dosage amount, and/or administering a SMAD2 agonist to a subject that is heterozygous or homozygous for the SMAD2 variant nucleic acid molecule; wherein the presence the SMAD2 variant nucleic acid molecule indicates the subject has an increased risk of developing inflammatory bowel disease.

The present disclosure also provides methods of identifying a subject having an increased risk of developing inflammatory bowel disease, the methods comprising: determining or having determined the presence or absence of a SMAD2 variant nucleic acid molecule in a biological sample obtained from the subject; wherein: when the subject is SMAD2 reference, then the subject has a decreased risk of developing inflammatory bowel disease; and when the subject is heterozygous or homozygous for the SMAD2 variant nucleic acid molecule, then the subject has an increased risk of developing inflammatory bowel disease.

The present disclosure also provides inflammatory bowel disease therapeutic agents for use in the treatment or prevention of inflammatory bowel disease in a subject having a SMAD2 variant nucleic acid molecule.

The present disclosure also provides SMAD2 agonists for use in the treatment or prevention of inflammatory bowel disease in a subject that is SMAD2 heterozygous or homozygous for a SMAD2 variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows representative associations between IDO2 variants and Crohn's disease.

FIG. 2 shows representative associations between IDO2 variants and Ulcerative Colitis.

FIG. 3 shows representative associations between SMAD2 variants and Ulcerative Colitis.

FIG. 4 shows representative associations between SMAD2 variants and Inflammatory Bowel Disease (IBD).

FIG. 5 shows representative associations between SMAD2 variants and Crohn's disease.

FIG. 6 shows pedigree trees of probands with likely pathogenic variants in IDO/SMAD2.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that rare/common IDO2 variant nucleic acid molecules (whether these variants are homozygous or heterozygous in a particular subject) are associated with an increased risk of developing IBD. It is believed that IDO2 variant nucleic acid molecules have not been associated with IBD in humans. Therefore, subjects that are IDO2 heterozygous or homozygous for an IDO2 variant nucleic acid molecule may be treated with an IDO2 agonist such that IBD is inhibited or prevented, the symptoms thereof are reduced or prevented, and/or development of symptoms is repressed or prevented. It is also believed that such subjects having IBD may further be treated with one or more IBD therapeutic agents. In addition, the present disclosure provides methods of leveraging the presence or absence of IDO2 variant nucleic acid molecules in subjects to identify or stratify risk in such subjects of developing IBD, or to diagnose subjects as having an increased risk of developing IBD.

For purposes of the present disclosure, any particular subject, such as a human, can be categorized as having one of three IDO2 genotypes: i) IDO2 reference; ii) heterozygous for an IDO2 variant nucleic acid molecule; or iii) homozygous for an IDO2 variant nucleic acid molecule. A subject is IDO2 reference when the subject does not have a copy of an IDO2 variant nucleic acid molecule. A subject is heterozygous for an IDO2 variant nucleic acid molecule when the subject has a single copy of an IDO2 variant nucleic acid molecule. A subject is homozygous for an IDO2 variant nucleic acid molecule when the subject has two copies of an IDO2 variant nucleic acid molecule.

In any of the embodiments described herein, the IDO2 variant nucleic acid molecule can be any nucleic acid molecule (such as, a genomic nucleic acid molecule, an mRNA molecule, or a cDNA molecule produced from an mRNA molecule) encoding an IDO2variant polypeptide having a partial loss-of-function, a complete loss-of-function, a predicted partial loss-of-function, or a predicted complete loss-of-function. A subject who has an IDO2 polypeptide having a partial loss-of-function (or predicted partial loss-of-function) is hypomorphic for IDO2. In some embodiments, the IDO2 variant nucleic acid molecule results in decreased or aberrant expression or activity of IDO2 mRNA or polypeptide. In some embodiments, the IDO2 variant nucleic acid molecule is associated with a reduced in vitro response to IDO2 ligands compared with reference IDO2. In some embodiments, the IDO2 variant nucleic acid molecule is a splice-site variant, a stop-gain variant, a start-loss variant, a stop-loss variant, a frameshift variant, an in-frame indel variant, or a variant that encodes a truncated IDO2 variant polypeptide. In some embodiments, the IDO2 variant nucleic acid molecule is a missense variant nucleic acid molecule. In some embodiments, the IDO2 variant nucleic acid molecule comprises a single nucleotide polymorphism (SNP). In some embodiments, the IDO2 variant nucleic acid molecule comprises a variation in a coding region. In some embodiments, the IDO2 variant nucleic acid molecule does not comprise a variation in a non-coding region, except for a splice acceptor region (two bases before the start of any exon except the first). In some embodiments, the IDO2 variant nucleic acid molecule results or is predicted to result in a premature truncation of an IDO2 polypeptide compared to the reference IDO2. In some embodiments, the IDO2 variant nucleic acid molecule is a variant that is predicted to be damaging to the protein function (and hence, in this case, protective to the human) by in vitro prediction algorithms such as Polyphen, SIFT, or similar algorithms. In some embodiments, the IDO2 variant nucleic acid molecule is a variant that causes or is predicted to cause a nonsynonymous amino acid substitution in an IDO2 nucleic acid molecule and whose allele frequency is less than 1/100 alleles in the population from which the subject is selected. In some embodiments, the IDO2 variant nucleic acid molecule is any rare missense variant (allele frequency<0.1%; or 1 in 1,000 alleles), or any splice-site, stop-gain, start-loss, stop-loss, frameshift, or in-frame indel, or other frameshift IDO2 variant.

In any of the embodiments described herein, the IDO2 variant genomic nucleic acid molecule may include one or more variations at any of the positions of chromosome 8 (i.e., positions 39,934,651-40,016,392) using the nucleotide sequence of the IDO2 reference genomic nucleic acid molecule in the GRCh38/hg38 human genome assembly (see, ENSG00000188676.15, ENST00000502986.4, RefSeq NM_194294 annotated in the Ensembl database (URL: world wide web at “https://useast.ensembl.org/Homo_sapiens/Gene/Summary? g=ENSG 00000188676; r=8:39934614-40016397; mobileredirect=no”)) as a reference sequence. The sequences provided in these transcripts for the IDO2 genomic nucleic acid molecule are only exemplary sequences. Other sequences for the IDO2 genomic nucleic acid molecule are also possible.

In any of the embodiments described herein, the IDO2 variant nucleic acid molecule may comprise any one or more of the following genetic variations in the genomic nucleic acid molecule (referring to the chromosome: positions set forth in the GRCh38/hg38 human genome assembly): 8:39989741:TG:T (Ala 191fs), 8:39989787:C:T, or 8:39989793:A:T, or an mRNA molecule produced therefrom, or a cDNA molecule produced from the mRNA molecule.

For subjects that are genotyped or determined to be IDO2 reference, such subjects have a decreased risk of developing IBD (compared to subjects that are heterozygous or homozygous for an IDO2 variant nucleic acid molecule). For subjects that are genotyped or determined to be IDO2 heterozygous or homozygous for an IDO2 variant nucleic acid molecule, such subjects have an increased risk of developing IBD and can be treated with an IDO2 agonist.

It has been observed in accordance with the present disclosure that rare/common SMAD2 variant nucleic acid molecules (whether these variants are homozygous or heterozygous in a particular subject) are associated with an increased risk of developing IBD. It is believed that SMAD2 variant nucleic acid molecules have not been associated with IBD in humans. Therefore, subjects that are SMAD2 heterozygous or homozygous for a SMAD2 variant nucleic acid molecule may be treated with a SMAD2 agonist such that IBD is inhibited or prevented, the symptoms thereof are reduced or prevented, and/or development of symptoms is repressed or prevented. It is also believed that such subjects having IBD may further be treated with one or more IBD therapeutic agents. In addition, the present disclosure provides methods of leveraging the presence or absence of SMAD2 variant nucleic acid molecules in subjects to identify or stratify risk in such subjects of developing IBD, or to diagnose subjects as having an increased risk of developing IBD.

For purposes of the present disclosure, any particular subject, such as a human, can be categorized as having one of three SMAD2 genotypes: i) SMAD2 reference; ii) heterozygous for a SMAD2 variant nucleic acid molecule; or iii) homozygous for a SMAD2 variant nucleic acid molecule. A subject is SMAD2 reference when the subject does not have a copy of a SMAD2 variant nucleic acid molecule. A subject is heterozygous for a SMAD2 variant nucleic acid molecule when the subject has a single copy of a SMAD2 variant nucleic acid molecule. A subject is homozygous for a SMAD2 variant nucleic acid molecule when the subject has two copies of a SMAD2 variant nucleic acid molecule.

In any of the embodiments described herein, the SMAD2 variant nucleic acid molecule can be any nucleic acid molecule (such as, a genomic nucleic acid molecule, an mRNA molecule, or a cDNA molecule produced from an mRNA molecule) encoding a SMAD2 variant polypeptide having a partial loss-of-function, a complete loss-of-function, a predicted partial loss-of-function, or a predicted complete loss-of-function. A subject who has a SMAD2 polypeptide having a partial loss-of-function (or predicted partial loss-of-function) is hypomorphic for SMAD2. In some embodiments, the SMAD2 variant nucleic acid molecule results in decreased or aberrant expression or activity of SMAD2 mRNA or polypeptide. In some embodiments, the SMAD2 variant nucleic acid molecule is associated with a reduced in vitro response to SMAD2 ligands compared with reference SMAD2. In some embodiments, the SMAD2 variant nucleic acid molecule is a splice-site variant, a stop-gain variant, a start-loss variant, a stop-loss variant, a frameshift variant, an in-frame indel variant, or a variant that encodes a truncated SMAD2 variant polypeptide. In some embodiments, the SMAD2 variant nucleic acid molecule is a missense variant nucleic acid molecule. In some embodiments, the SMAD2 variant nucleic acid molecule comprises a single nucleotide polymorphism (SNP). In some embodiments, the SMAD2 variant nucleic acid molecule comprises a variation in a coding region. In some embodiments, the SMAD2 variant nucleic acid molecule does not comprise a variation in a non-coding region, except for a splice acceptor region (two bases before the start of any exon except the first). In some embodiments, the SMAD2 variant nucleic acid molecule results or is predicted to result in a premature truncation of a SMAD2 polypeptide compared to the reference SMAD2. In some embodiments, the SMAD2 variant nucleic acid molecule is a variant that is predicted to be damaging to the protein function (and hence, in this case, protective to the human) by in vitro prediction algorithms such as Polyphen, SIFT, or similar algorithms. In some embodiments, the SMAD2 variant nucleic acid molecule is a variant that causes or is predicted to cause a nonsynonymous amino acid substitution in a SMAD2 nucleic acid molecule and whose allele frequency is less than 1/100 alleles in the population from which the subject is selected. In some embodiments, the SMAD2 variant nucleic acid molecule is any rare missense variant (allele frequency<0.1%; or 1 in 1,000 alleles), or any splice-site, stop-gain, start-loss, stop-loss, frameshift, or in-frame indel, or other frameshift SMAD2 variant.

In any of the embodiments described herein, the SMAD2 variant genomic nucleic acid molecule may include one or more variations at any of the positions of chromosome 18 (i.e., positions 47,808,957-47,930,659) using the nucleotide sequence of the SMAD2 reference genomic nucleic acid molecule in the GRCh38/hg38 human genome assembly (see, ENSG00000175387.16, ENST00000262160.11, RefSeq NM_005901 annotated in the Ensembl database (URL: world wide web at “http://useast.ensembl.org/Homo_sapiens/Gene/Summary? g=ENSG 00000175387; r=18: 47808957-47931146”)) as a reference sequence. The sequences provided in these transcripts for the SMAD2 genomic nucleic acid molecule are only exemplary sequences. Other sequences for the SMAD2 genomic nucleic acid molecule are also possible.

In any of the embodiments described herein, the SMAD2 variant nucleic acid molecule may comprise any one or more of the following genetic variations in the genomic nucleic acid molecule (referring to the chromosome: positions set forth in the GRCh38/hg38 human genome assembly): 18:47869435:AC:A (Arg109SerfsTer54), 18:47845485:C:A, or 18:47896588:G:A, or an mRNA molecule produced therefrom, or a cDNA molecule produced from the mRNA molecule.

For subjects that are genotyped or determined to be SMAD2 reference, such subjects have a decreased risk of developing IBD (compared to subjects that are heterozygous or homozygous for a SMAD2 variant nucleic acid molecule). For subjects that are genotyped or determined to be SMAD2 heterozygous or homozygous for a SMAD2variant nucleic acid molecule, such subjects have an increased risk of developing IBD and can be treated with a SMAD2 agonist.

In any of the embodiments described herein, the subject in whom IBD is prevented by administering an IDO2 agonist or a SMAD2 agonist may be anyone at risk for developing IBD including, but not limited to, subjects with a genetic predisposition for developing IBD. In some embodiments, administering an IDO2 agonist or a SMAD2 agonist to a subject having IBD may be carried out to prevent development of another occurrence of IBD in a subject who has already had IBD. In any of the embodiments described herein, the methods can be used to improve IBD.

In any of the embodiments described herein, the IDO2 predicted loss-of-function polypeptide can be any IDO2 polypeptide having a partial loss-of-function, a complete loss-of-function, a predicted partial loss-of-function, or a predicted complete loss-of-function.

Any one or more (i.e., any combination) of the IDO2 variant nucleic acid molecules described herein can be used within any of the methods described herein to determine whether a subject has an increased or decreased risk of developing IBD. The combinations of particular variants can form a mask used for statistical analysis of the particular correlation of IDO2 and an increased or decreased risk of developing IBD. In some embodiments, the mask used for statistical analysis of the particular correlation of IDO2 and an increased or decreased risk of developing IBD can exclude any one or more of these IDO2 variant nucleic acid molecules described herein.

In any of the embodiments described herein, the SMAD2 predicted loss-of-function polypeptide can be any SMAD2 polypeptide having a partial loss-of-function, a complete loss-of-function, a predicted partial loss-of-function, or a predicted complete loss-of-function.

Any one or more (i.e., any combination) of the SMAD2 variant nucleic acid molecules described herein can be used within any of the methods described herein to determine whether a subject has an increased or decreased risk of developing IBD. The combinations of particular variants can form a mask used for statistical analysis of the particular correlation of SMAD2 and an increased or decreased risk of developing IBD. In some embodiments, the mask used for statistical analysis of the particular correlation of SMAD2 and an increased or decreased risk of developing IBD can exclude any one or more of these SMAD2 variant nucleic acid molecules described herein.

In any of the embodiments described herein, the subject can have IBD. In any of the embodiments described herein, the subject can be at risk of developing IBD. In any of the embodiments described herein, the methods can be used to treat a complication or co-morbidity of IBD, or reduce the risk of developing the same.

The present disclosure provides methods of treating a subject having IBD or at risk of developing IBD, the methods comprising administering an IDO2 agonist to the subject.

In any of the methods of treatment described herein, the subject being treated may comprise an IDO2 variant nucleic acid molecule. In some embodiments, the subject being treated is heterozygous for the IDO2 variant nucleic acid molecule. In some embodiments, the subject being treated is homozygous for the IDO2 variant nucleic acid molecule. In some embodiments, the subject being treated is IDO2 reference. The IDO2 variant nucleic acid molecule can be any of the IDO2 variant nucleic acid molecules disclosed herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

In some embodiments, the methods of treatment further comprise detecting the presence or absence of an IDO2 variant nucleic acid molecule in a biological sample from the subject. In some embodiments, the IDO2 variant nucleic acid molecule can be any of the IDO2 variant nucleic acid molecules disclosed herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

The present disclosure also provides methods of treating a subject with an IBD therapeutic agent, wherein the subject has IBD or is at risk of developing IBD. The methods comprise determining whether the subject has an IDO2 variant nucleic acid molecule by obtaining or having obtained a biological sample from the subject, and performing or having performed a sequence analysis on the biological sample to determine if the subject has a genotype comprising the IDO2 variant nucleic acid molecule. In embodiments where the subject is IDO2 reference, the methods further comprise administering or continuing to administer the IBD therapeutic agent in an amount that is the same as a standard dosage amount to the subject to the subject. In embodiments where the subject is heterozygous or homozygous for the IDO2 variant nucleic acid molecule, the methods further comprise administering or continuing to administer the IBD therapeutic agent in an amount that is the same as or less than a standard dosage amount to the subject, and/or administering an IDO2 agonist to the subject. The presence of an IDO2 variant nucleic acid molecule indicates the subject has an increased risk of developing IBD. In some embodiments, the subject is IDO2 reference. In some embodiments, the subject is heterozygous for an IDO2 variant nucleic acid molecule. In some embodiments, the subject is homozygous for an IDO2 variant nucleic acid molecule. In any of the embodiments described herein, the IDO2 agonist is an example of an IBD therapeutic agent. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

For subjects that are genotyped or determined to be IDO2 heterozygous or homozygous for an IDO2 variant nucleic acid molecule, such subjects can be administered an IDO2 agonist, as described herein.

In some embodiments, when the subject is IDO2 reference, the subject is administered an IBD therapeutic agent in an amount that is the same as a standard dosage amount. In some embodiments, when the subject is heterozygous or homozygous for an IDO2 variant nucleic acid molecule, the subject is administered an IBD therapeutic agent in an amount that is the same as or less than a standard dosage amount, and/or an IDO2 agonist.

In some embodiments, the treatment methods comprise detecting the presence or absence of a decrease in the expression of an IDO2 variant mRNA or polypeptide in a biological sample from the subject. In some embodiments, when the subject does not have a decrease in the expression of an IDO2 variant mRNA or polypeptide, the subject is administered an IBD therapeutic agent in an amount that is the same as a standard dosage amount. In some embodiments, when the subject has a decrease in the expression of an IDO2 variant mRNA or polypeptide, the subject is administered an IBD therapeutic agent in a standard dosage amount or less than a standard dosage amount, and/or an IDO2 agonist.

The present disclosure also provides methods of treating a subject with an IBD therapeutic agent, wherein the subject has IBD or is at risk of developing IBD. The methods comprise determining whether the subject has a decrease in the expression of an IDO2 variant mRNA or polypeptide by obtaining or having obtained a biological sample from the subject, and performing or having performed an assay on the biological sample to determine if the subject a decrease in the expression of an IDO2 variant mRNA or polypeptide. In embodiments where the subject does not have a decrease in the expression of an IDO2 variant mRNA or polypeptide, the methods further comprise administering or continuing to administer the IBD therapeutic agent in an amount that is the same as a standard dosage amount to the subject. In embodiments where the subject has a decrease in the expression of an IDO2 variant mRNA or polypeptide, the methods further comprise administering or continuing to administer the IBD therapeutic agent in an amount that is the same as or less than a standard dosage amount to the subject, and/or administering an IDO2 agonist to the subject. The presence of a decrease in the expression of an IDO2 variant mRNA or polypeptide indicates the subject has an increased risk of developing IBD. In some embodiments, the subject has a decrease in the expression of an IDO2 variant mRNA or polypeptide. In some embodiments, the subject does not have a decrease in the expression of an IDO2 variant mRNA or polypeptide. In any of the embodiments described herein, the IDO2 agonist is an example of an IBD therapeutic agent. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

In some embodiments, the treatment methods comprise detecting the presence or absence of an IDO2 variant polypeptide in a biological sample from the subject. In some embodiments, when the subject does not have an IDO2 variant polypeptide, the subject is administered an IBD therapeutic agent in an amount that is the same a standard dosage amount. In some embodiments, when the subject has an IDO2 variant polypeptide, the subject is administered an IBD therapeutic agent in an amount that is the same as or less than a standard dosage amount, and/or is administered an IDO2 agonist.

The present disclosure also provides methods of treating a subject with an IBD therapeutic agent, wherein the subject has IBD or is at risk of developing IBD. The methods comprise determining whether the subject has an IDO2 variant polypeptide by obtaining or having obtained a biological sample from the subject and performing or having performed an assay on the biological sample to determine if the subject has an IDO2 variant polypeptide. When the subject does not have an IDO2 variant polypeptide, the subject is administered the IBD therapeutic agent in an amount that is the same as a standard dosage amount. When the subject has an IDO2 variant polypeptide, the subject is administered the IBD therapeutic agent in an amount that is the same as or less than a standard dosage amount, and/or is administered an IDO2 agonist. The presence of an IDO2 variant polypeptide indicates the subject has an increased risk of developing IBD. In some embodiments, the subject has an IDO2 variant polypeptide. In some embodiments, the subject does not have an IDO2 variant polypeptide.

The present disclosure also provides methods of preventing a subject from developing IBD by administering an IBD therapeutic agent. In some embodiments, the method comprises determining whether the subject has an IDO2 variant polypeptide by obtaining or having obtained a biological sample from the subject and performing or having performed an assay on the biological sample to determine if the subject has an IDO2 variant polypeptide. When the subject does not have an IDO2 variant polypeptide, the subject is administered the IBD therapeutic agent in an amount that is the same as a standard dosage amount. When the subject has an IDO2 variant polypeptide, the subject is administered the IBD therapeutic agent in an amount that is the same as or less than a standard dosage amount, and/or is administered an IDO2 agonist. The presence of an IDO2 variant polypeptide indicates the subject has an increased risk of developing IBD. In some embodiments, the subject has an IDO2 variant polypeptide. In some embodiments, the subject does not have an IDO2 variant polypeptide.

In some embodiments, the IDO2 agonist is a small molecule. In some embodiments, the small molecule is low molecular weight (<900 daltons) organic compound.

The present disclosure provides methods of treating a subject having IBD or at risk of developing IBD, the methods comprising administering a SMAD2 agonist to the subject.

In any of the methods of treatment described herein, the subject being treated may comprise a SMAD2 variant nucleic acid molecule. In some embodiments, the subject being treated is heterozygous for the SMAD2 variant nucleic acid molecule. In some embodiments, the subject being treated is homozygous for the SMAD2 variant nucleic acid molecule. In some embodiments, the subject being treated is SMAD2 reference. The SMAD2 variant nucleic acid molecule can be any of the SMAD2 variant nucleic acid molecules disclosed herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

In some embodiments, the methods of treatment further comprise detecting the presence or absence of a SMAD2 variant nucleic acid molecule in a biological sample from the subject. In some embodiments, the SMAD2 variant nucleic acid molecule can be any of the SMAD2 variant nucleic acid molecules disclosed herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

The present disclosure also provides methods of treating a subject with an IBD therapeutic agent, wherein the subject has IBD or is at risk of developing IBD. The methods comprise determining whether the subject has a SMAD2 variant nucleic acid molecule by obtaining or having obtained a biological sample from the subject, and performing or having performed a sequence analysis on the biological sample to determine if the subject has a genotype comprising the SMAD2 variant nucleic acid molecule. In embodiments where the subject is SMAD2 reference, the methods further comprise administering or continuing to administer the IBD therapeutic agent in an amount that is the same as a standard dosage amount to the subject to the subject. In embodiments where the subject is heterozygous or homozygous for the SMAD2 variant nucleic acid molecule, the methods further comprise administering or continuing to administer the IBD therapeutic agent in an amount that is the same as or less than a standard dosage amount to the subject, and/or administering a SMAD2 agonist to the subject. The presence of a SMAD2 variant nucleic acid molecule indicates the subject has an increased risk of developing IBD. In some embodiments, the subject is SMAD2 reference. In some embodiments, the subject is heterozygous for a SMAD2 variant nucleic acid molecule. In some embodiments, the subject is homozygous for a SMAD2 variant nucleic acid molecule. In any of the embodiments described herein, the SMAD2 agonist is an example of an IBD therapeutic agent. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

For subjects that are genotyped or determined to be SMAD2 heterozygous or homozygous for a SMAD2 variant nucleic acid molecule, such subjects can be administered a SMAD2 agonist, as described herein.

In some embodiments, when the subject is SMAD2 reference, the subject is administered an IBD therapeutic agent in an amount that is the same as a standard dosage amount. In some embodiments, when the subject is heterozygous or homozygous for a SMAD2 variant nucleic acid molecule, the subject is administered an IBD therapeutic agent in an amount that is the same as or less than a standard dosage amount, and/or a SMAD2 agonist.

In some embodiments, the treatment methods comprise detecting the presence or absence of a decrease in the expression of a SMAD2 variant mRNA or polypeptide in a biological sample from the subject. In some embodiments, when the subject does not have a decrease in the expression of a SMAD2 variant mRNA or polypeptide, the subject is administered an IBD therapeutic agent in an amount that is the same as a standard dosage amount. In some embodiments, when the subject has a decrease in the expression of a SMAD2 variant mRNA or polypeptide, the subject is administered an IBD therapeutic agent in a standard dosage amount or less than a standard dosage amount, and/or a SMAD2 agonist.

The present disclosure also provides methods of treating a subject with an IBD therapeutic agent, wherein the subject has IBD or is at risk of developing IBD. The methods comprise determining whether the subject has a decrease in the expression of a SMAD2 variant mRNA or polypeptide by obtaining or having obtained a biological sample from the subject, and performing or having performed an assay on the biological sample to determine if the subject a decrease in the expression of a SMAD2 variant mRNA or polypeptide. In embodiments where the subject does not have a decrease in the expression of a SMAD2 variant mRNA or polypeptide, the methods further comprise administering or continuing to administer the IBD therapeutic agent in an amount that is the same as a standard dosage amount to the subject. In embodiments where the subject has a decrease in the expression of a SMAD2 variant mRNA or polypeptide, the methods further comprise administering or continuing to administer the IBD therapeutic agent in an amount that is the same as or less than a standard dosage amount to the subject, and/or administering a SMAD2 agonist to the subject. The presence of a decrease in the expression of a SMAD2 variant mRNA or polypeptide indicates the subject has an increased risk of developing IBD. In some embodiments, the subject has a decrease in the expression of a SMAD2 variant mRNA or polypeptide. In some embodiments, the subject does not have a decrease in the expression of a SMAD2 variant mRNA or polypeptide. In any of the embodiments described herein, the SMAD2 agonist is an example of an IBD therapeutic agent. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

In some embodiments, the treatment methods comprise detecting the presence or absence of a SMAD2 variant polypeptide in a biological sample from the subject. In some embodiments, when the subject does not have a SMAD2 variant polypeptide, the subject is administered an IBD therapeutic agent in an amount that is the same a standard dosage amount. In some embodiments, when the subject has a SMAD2 variant polypeptide, the subject is administered an IBD therapeutic agent in an amount that is the same as or less than a standard dosage amount, and/or is administered a SMAD2 agonist.

The present disclosure also provides methods of treating a subject with an IBD therapeutic agent, wherein the subject has IBD or is at risk of developing IBD. The methods comprise determining whether the subject has a SMAD2 variant polypeptide by obtaining or having obtained a biological sample from the subject and performing or having performed an assay on the biological sample to determine if the subject has a SMAD2 variant polypeptide. When the subject does not have a SMAD2 variant polypeptide, the subject is administered the IBD therapeutic agent in an amount that is the same as a standard dosage amount. When the subject has a SMAD2 variant polypeptide, the subject is administered the IBD therapeutic agent in an amount that is the same as or less than a standard dosage amount, and/or is administered a SMAD2 agonist. The presence of a SMAD2 variant polypeptide indicates the subject has an increased risk of developing IBD. In some embodiments, the subject has a SMAD2 variant polypeptide. In some embodiments, the subject does not have a SMAD2 variant polypeptide.

The present disclosure also provides methods of preventing a subject from developing IBD by administering an IBD therapeutic agent. In some embodiments, the method comprises determining whether the subject has a SMAD2 variant polypeptide by obtaining or having obtained a biological sample from the subject and performing or having performed an assay on the biological sample to determine if the subject has a SMAD2 variant polypeptide. When the subject does not have a SMAD2 variant polypeptide, the subject is administered the IBD therapeutic agent in an amount that is the same as a standard dosage amount. When the subject has a SMAD2 variant polypeptide, the subject is administered the IBD therapeutic agent in an amount that is the same as or less than a standard dosage amount, and/or is administered a SMAD2 agonist. The presence of a SMAD2 variant polypeptide indicates the subject has an increased risk of developing IBD. In some embodiments, the subject has a SMAD2 variant polypeptide. In some embodiments, the subject does not have a SMAD2 variant polypeptide.

In some embodiments, the SMAD2 agonist is a small molecule. In some embodiments, the small molecule is low molecular weight (<900 daltons) organic compound.

In some embodiments, the IBD therapeutic agents include, but are not limited to, an aminosalicylate (such as COLAZAL® (balsalazide), ASACOL®, APRISO®, LIALDA®, and PENTASA® (mesalamine), DIPENTUM® (olsalazine), and AZULFIDINE® (sulfasalazine)); a corticosteroid; an immune modifying agent (such as IMURAN® (azathioprine), RHEUMATREX® (methotrexate), and PURINETHOL® (6-mercaptopurine (6-MP)); an antibody (such as REMICADE® (infliximab), RENFLEXIS® (infliximab-abda), INFLECTRA® (infliximab-dyyb), HUMIRA® (dalimumab), AMJEVITA® (adalimumab-atto), CIMZIA® (certolizumab), TYSABRI® (natalizumab), ENTYVIO® (vedolizumab), STELARA® (ustekinumab), SIMPONI® and SIMPONI® ARIA (golimumab)), and a small molecule (such as XELJANZ® (tofacitinib), RINVOQ® (upadacitinib), and ZEPOSIA® (ozanimod)), or any combination thereof. In some embodiments, the IBD therapeutic agents include, but are not limited to, an aminosalicylate (such as balsalazide, mesalamine, olsalazine, and sulfasalazine); a corticosteroid; an immune modifying agent (such as azathioprine, methotrexate, and 6-mercaptopurine (6-MP)); an antibody (such as infliximab, infliximab-abda, infliximab-dyyb, dalimumab, adalimumab-atto, certolizumab, natalizumab, vedolizumab, ustekinumab, golimumab); and a small molecule (such as tofacitinib, upadacitinib, and ozanimod), or any combination thereof. Additional IBD therapies include any therapy used to reduce or manage IBD risk factors. In some embodiments, the IBD therapeutic agent can be combined with an IDO2 agonist or a SMAD2 agonist. These treatment therapies may be delayed or avoided altogether by treatment with an IDO2 agonist or a SMAD2 agonist as described herein.

In some embodiments, the IBD therapeutic agent can be combined with an IDO2 agonist or a SMAD2 agonist. These treatment therapies may be delayed or avoided altogether by treatment with an IDO2 agonist or a SMAD2 agonist agonist as described herein.

In some embodiments, the dose of the IBD therapeutic agents that treat, prevent, or inhibit IBD can be decreased by about 10%, by about 20%, by about 30%, by about 40%, by about 50%, by about 60%, by about 70%, by about 80%, or by about 90% for subjects that are heterozygous or homozygous for an IDO2 variant nucleic acid molecule or a SMAD2 variant nucleic acid molecule (i.e., a less than the standard dosage amount) compared to subjects that are IDO2 reference or SMAD2 reference (who may receive a standard dosage amount), respectively. In some embodiments, the dose of the IBD therapeutic agents that treat, prevent, or inhibit IBD can be decreased by about 10%, by about 20%, by about 30%, by about 40%, or by about 50%. In some embodiments, the dose of the IBD therapeutic agents that treat, prevent, or inhibit IBD can be decreased by about 10%, by about 20%, by about 30%, by about 40%, by about 50%, by about 60%, by about 70%, by about 80%, or by about 90% for subjects that are homozygous for an IDO2 variant nucleic acid molecule or a SMAD2 variant nucleic acid molecule compared to subjects that are heterozygous for an IDO2 variant nucleic acid molecule or a SMAD2 variant nucleic acid molecule, respectively. In addition, subjects that are heterozygous or homozygous for an IDO2 variant nucleic acid molecule or a SMAD2 variant nucleic acid moleculecan be administered the IBD therapeutic agents less frequently compared to subjects that are IDO2 reference or SMAD2 reference, respectively.

Administration of the IBD therapeutic agents that treat, prevent, or inhibit IBD and/or IDO2 agonists or SMAD2 agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the IBD therapeutic agents and/or IDO2 agonists or SMAD2 agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in IBD, a decrease/reduction in the severity of IBD (such as, for example, a reduction or inhibition of development of IBD), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to IBD, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of IBD development/progression (such as, for example, a complete or partial avoidance/inhibition or a delay), and an increased survival time of the affected host animal, following administration of a therapeutic protocol. Treatment of IBD encompasses the treatment of a subject already diagnosed as having any form of IBD at any clinical stage or manifestation, the delay of the onset or evolution or aggravation or deterioration of the symptoms or signs of IBD, and/or preventing and/or reducing the severity of IBD.

Detecting the presence or absence of an IDO2 variant nucleic acid molecule or a SMAD2 variant nucleic acid molecule in a biological sample from a subject and/or determining whether a subject has an IDO2 variant nucleic acid molecule or a SMAD2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

Detecting a decrease in the expression of an IDO2 variant mRNA or polypeptide or a SMAD2 variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

Detecting the presence or absence of an IDO2 variant polypeptide or a SMAD2 variant polypeptide in a biological sample from a subject and/or determining whether a subject has an IDO2 variant polypeptide or a SMAD2 variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

The present disclosure also provides methods of identifying a subject having an increased risk of developing IBD. In some embodiments, the method comprises determining or having determined in a biological sample obtained from the subject the presence or absence of an IDO2 variant nucleic acid molecule (such as a genomic nucleic acid molecule, mRNA molecule, and/or cDNA molecule). When the subject lacks an IDO2 variant nucleic acid molecule (i.e., the subject is genotypically categorized as IDO2 reference), then the subject has a decreased risk of developing IBD (compared to subjects that are heterozygous or homozygous for an IDO2 variant nucleic acid molecule). When the subject has an IDO2 variant nucleic acid molecule (i.e., the subject is heterozygous or homozygous for an IDO2 variant nucleic acid molecule), then the subject has an increased risk of developing IBD. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

Having two copies of an IDO2 variant nucleic acid molecule may result in a greater risk of a subject for developing IBD than having a single copy of an IDO2 variant nucleic acid molecule. Without intending to be limited to any particular theory or mechanism of action, it is believed that a single copy of an IDO2 variant nucleic acid molecule (i.e., heterozygous for an IDO2 variant nucleic acid molecule) increases the risk of a subject for developing IBD and it is also believed that having two copies of an IDO2 variant nucleic acid molecule (i.e., homozygous for an IDO2 variant nucleic acid molecule) may increase the risk even more of a subject for developing IBD and may increase the risk of a subject developing IBD at a younger age, relative to a subject with a single copy.

In some embodiments, when a subject is identified as having an increased risk of developing IBD, the subject is administered an IBD therapeutic agent, and/or an IDO2 agonist, as described herein. For example, when the subject is IDO2 reference, and therefore has a decreased risk of developing IBD, the subject is administered an IBD therapeutic agent in an amount that is the same as a standard dosage amount. In some embodiments, when the subject is heterozygous or homozygous for an IDO2 variant nucleic acid molecule, the subject is administered the IBD therapeutic agent in an amount that is the same as or less than a standard dosage amount, and/or is administered an IDO2 agonist. In some embodiments, the subject is IDO2 reference. In some embodiments, the subject is heterozygous for an IDO2 variant nucleic acid molecule. In some embodiments, the subject is homozygous for an IDO2 variant nucleic acid molecule.

The present disclosure also provides methods of identifying a subject having an increased risk of developing IBD. In some embodiments, the method comprises determining or having determined in a biological sample obtained from the subject the presence or absence of a SMAD2 variant nucleic acid molecule (such as a genomic nucleic acid molecule, mRNA molecule, and/or cDNA molecule). When the subject lacks a SMAD2 variant nucleic acid molecule (i.e., the subject is genotypically categorized as SMAD2 reference), then the subject has a decreased risk of developing IBD (compared to subjects that are heterozygous or homozygous for a SMAD2 variant nucleic acid molecule). When the subject has a SMAD2 variant nucleic acid molecule (i.e., the subject is heterozygous or homozygous for a SMAD2 variant nucleic acid molecule), then the subject has an increased risk of developing IBD. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

Having two copies of a SMAD2 variant nucleic acid molecule may result in a greater risk of a subject for developing IBD than having a single copy of a SMAD2 variant nucleic acid molecule. Without intending to be limited to any particular theory or mechanism of action, it is believed that a single copy of a SMAD2 variant nucleic acid molecule (i.e., heterozygous for a SMAD2 variant nucleic acid molecule) increases the risk of a subject for developing IBD and it is also believed that having two copies of a SMAD2 variant nucleic acid molecule (i.e., homozygous for a SMAD2 variant nucleic acid molecule) may increase the risk even more of a subject for developing IBD, relative to a subject with a single copy.

In some embodiments, when a subject is identified as having an increased risk of developing IBD, the subject is administered an IBD therapeutic agent, and/or a SMAD2 agonist, as described herein. For example, when the subject is SMAD2 reference, and therefore has a decreased risk of developing IBD, the subject is administered an IBD therapeutic agent in an amount that is the same as a standard dosage amount. In some embodiments, when the subject is heterozygous or homozygous for a SMAD2 variant nucleic acid molecule, the subject is administered the IBD therapeutic agent in an amount that is the same as or less than a standard dosage amount, and/or is administered a SMAD2 agonist. In some embodiments, the subject is SMAD2 reference. In some embodiments, the subject is heterozygous for a SMAD2 variant nucleic acid molecule. In some embodiments, the subject is homozygous for a SMAD2 variant nucleic acid molecule.

Determining whether a subject has an IDO2 variant nucleic acid molecule or a SMAD2 variant nucleic acid molecule in a biological sample from a subject and/or determining whether a subject has an IDO2 variant nucleic acid molecule or a SMAD2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of determining a subject's aggregate burden, or risk score, of having two or more IDO2 variant nucleic acid molecules or SMAD2 variant nucleic acid molecules, and/or two or more IDO2 variant polypeptides or SMAD2 variant polypeptides associated with an increased risk of developing IBD. The aggregate burden is the sum of two or more genetic variants that can be carried out in an association analysis with IBD. In some embodiments, the subject is homozygous for one or more IDO2 variant nucleic acid molecules or SMAD2 variant nucleic acid molecules associated with an increased risk of developing IBD. In some embodiments, the subject is heterozygous for one or more IDO2 variant nucleic acid molecules or SMAD2 variant nucleic acid molecules associated with an increased risk of developing IBD. When the subject has a lower aggregate burden, the subject has a decreased risk of developing IBD, and the subject is administered or continued to be administered the IBD therapeutic agent in an amount that is the same as a standard dosage amount. When the subject has a higher aggregate burden, the subject has an increased risk of developing IBD and the subject is administered or continued to be administered the IBD therapeutic agent in an amount that is the same as or less than a standard dosage amount, and/or an IDO2 agonist or a SMAD2 agonist. The higher the aggregate burden, the higher the risk of developing IBD.

In some embodiments, a subject's aggregate burden of having any two or more IDO2 variant nucleic acid molecules or SMAD2 variant nucleic acid molecules represents a weighted sum of a plurality of any of the IDO2 variant nucleic acid molecules or SMAD2 variant nucleic acid molecules, respectively. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the IDO2 gene or SMAD2 gene, where the genetic burden is the number of alleles multiplied by the association estimate with IBD or related outcome for each allele (e.g., a weighted polygenic burden score). In some embodiments, when the subject has an aggregate burden higher than a desired threshold score, the subject has an increased risk of developing IBD. In some embodiments, when the subject has an aggregate burden lower than a desired threshold score, the subject has a decreased risk of developing IBD.

In some embodiments, the aggregate burden may be divided into quintiles, e.g., top quintile, second quintile, intermediate quintile, fourth quintile, and bottom quintile, wherein the top quintile of aggregate burden corresponds to the highest risk group and the bottom quintile of aggregate burden corresponds to the lowest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with IBD in the top 10%, top 20%, top 30%, top 40%, or top 50% of p-value range for the association. In some embodiments, each of the identified genetic variants comprise the genetic variants having association with IBD with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with IBD with p-value of less than 5×10⁻⁸. In some embodiments, the identified genetic variants comprise genetic variants having association with IBD in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the top decile, quintile, or tertile in a reference population. The threshold of the aggregate burden can be determined on the basis of the nature of the intended practical application and the risk difference that would be considered meaningful for that practical application.

In embodiments where the aggregate burden is determined for IDO2 genetic variants associated with IBD, then the aggregate burden represents a subject's risk score for developing IBD. In some embodiments, the aggregate burden or risk score includes the IDO2variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule. In some embodiments, a subject's aggregate burden can be determined for IDO2 genetic variants associated with IBD in combination with additional genetic variants for other genes also associated with IBD to produce a polygenic risk score (PRS) for developing IBD. In some embodiments, the PRS includes the IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

In embodiments where the aggregate burden is determined for SMAD2 genetic variants associated with IBD, then the aggregate burden represents a subject's risk score for developing IBD. In some embodiments, the aggregate burden or risk score includes the SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule. In some embodiments, a subject's aggregate burden can be determined for SMAD2 genetic variants associated with IBD in combination with additional genetic variants for other genes also associated with IBD to produce a polygenic risk score (PRS) for developing IBD. In some embodiments, the PRS includes the SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

The present disclosure also provides methods of detecting the presence or absence of an IDO2 variant nucleic acid molecule or a SMAD2 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any IDO2 variant nucleic acid molecule or SMAD2 variant nucleic acid molecule, preliminary processing designed to isolate or enrich the biological sample for the genomic DNA can be employed. A variety of techniques may be used for this purpose. When detecting the level of any IDO2 variant nucleic acid molecules or SMAD2 variant nucleic acid molecules, different techniques can be used enrich the biological sample with mRNA molecules. Various methods to detect the presence or level of an mRNA molecule or the presence of a particular variant genomic DNA locus can be used.

In some embodiments, detecting an IDO2 variant nucleic acid molecule in a subject comprises performing a sequence analysis on a biological sample obtained from the subject to determine whether an IDO2 genomic nucleic acid molecule in the biological sample, and/or an IDO2 mRNA molecule in the biological sample, and/or an IDO2 cDNA molecule produced from an mRNA molecule in the biological sample, is present in the sample. In some embodiments, the methods detect the IDO2 variant genomic nucleic acid molecule that comprises the genetic variation 8:39989741:TG:T (Ala191fs), 8:39989787:C:T, or 8:39989793:A:T, or an mRNA molecule produced therefrom, or a cDNA molecule produced from the mRNA molecule.

In some embodiments, detecting a SMAD2 variant nucleic acid molecule in a subject comprises performing a sequence analysis on a biological sample obtained from the subject to determine whether a SMAD2 genomic nucleic acid molecule in the biological sample, and/or a SMAD2 mRNA molecule in the biological sample, and/or a SMAD2 cDNA molecule produced from an mRNA molecule in the biological sample, is present in the sample. In some embodiments, the methods detect the SMAD2 variant genomic nucleic acid molecule that comprises the genetic variation 18:47869435:AC:A (Arg109SerfsTer54), 18:47845485:C:A, or 18:47896588:G:A, or an mRNA molecule produced therefrom, or a cDNA molecule produced from the mRNA molecule.

In some embodiments, the methods of detecting the presence or absence of an IDO2 variant nucleic acid molecule or a SMAD2 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an IDO2 genomic nucleic acid molecule or mRNA molecule or a SMAD2 genomic nucleic acid molecule or mRNA molecule, and if mRNA, optionally reverse transcribing the mRNA into cDNA. Such assays can comprise, for example determining the identity of these positions of the particular IDO2 nucleic acid molecule or SMAD2 nucleic acid molecule, respectively. In some embodiments, the method is an in vitro method.

In some embodiments, the determining step, detecting step, or sequence analysis comprises sequencing at least a portion of the nucleotide sequence of the IDO2 genomic nucleic acid molecule, the IDO2 mRNA molecule, or the IDO2 cDNA molecule in the biological sample that comprises a genetic variation compared to the corresponding IDO2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an IDO2 genomic nucleic acid molecule is analyzed. In some embodiments, only an IDO2 mRNA is analyzed. In some embodiments, only an IDO2 cDNA obtained from the IDO2 mRNA is analyzed.

In some embodiments, the determining step, detecting step, or sequence analysis comprises sequencing at least a portion of the nucleotide sequence of the SMAD2 genomic nucleic acid molecule, the SMAD2 mRNA molecule, or the SMAD2 cDNA molecule in the biological sample that comprises a genetic variation compared to the corresponding SMAD2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a SMAD2 genomic nucleic acid molecule is analyzed. In some embodiments, only a SMAD2 mRNA is analyzed. In some embodiments, only a SMAD2 cDNA obtained from the SMAD2 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject. In some embodiments, the assay comprises contacting the biological sample with a primer or probe, such as an alteration-specific primer or alteration-specific probe, that specifically hybridizes to an IDO2 variant genomic sequence, variant mRNA sequence, or variant cDNA sequence and not the corresponding IDO2 reference sequence under stringent conditions and determining whether hybridization has occurred. In some embodiments, the assay comprises contacting the biological sample with a primer or probe, such as an alteration-specific primer or alteration-specific probe, that specifically hybridizes to a SMAD2 variant genomic sequence, variant mRNA sequence, or variant cDNA sequence and not the corresponding SMAD2 reference sequence under stringent conditions and determining whether hybridization has occurred.

In some embodiments, the determining step, detecting step, or sequence analysis comprises: a) amplifying at least a portion of the IDO2 nucleic acid molecule that encodes the IDO2 polypeptide; b) labeling the amplified nucleic acid molecule with a detectable label; c) contacting the labeled nucleic acid molecule with a support comprising an alteration-specific probe; and d) detecting the detectable label.

In some embodiments, the determining step, detecting step, or sequence analysis comprises: a) amplifying at least a portion of the SMAD2 nucleic acid molecule that encodes the SMAD2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an IDO2 variant genomic nucleic acid molecule, variant mRNA molecule, or variant cDNA molecule or a SMAD2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to IDO2 variant nucleic acid molecules or SMAD2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to IDO2 variant nucleic acid molecules or SMAD2 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the IDO2 variant nucleic acid molecules disclosed herein. The primers described herein can be used to amplify any IDO2 variant nucleic acid molecule, or a fragment thereof. The probes and primers described herein can be used to detect a nucleotide variation within any of the SMAD2 variant nucleic acid molecules disclosed herein. The primers described herein can be used to amplify any SMAD2 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding: an IDO2 reference genomic nucleic acid molecule or an IDO2 reference mRNA molecule, and/or an IDO2 reference cDNA molecule, or a SMAD2 reference genomic nucleic acid molecule, a SMAD2 reference mRNA molecule, and/or a SMAD2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, agonists, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410;

Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides IBD therapeutic agents that treat, prevent, or inhibit IBD for use in the treatment or prevention of IBD in a subject having an IDO2 variant nucleic acid molecule. Any of the IBD therapeutic agents that treat, prevent, or inhibit IBD described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

The present disclosure also provides uses of IBD therapeutic agents that treat, prevent, or inhibit IBD for use in the preparation of a medicament for treating or preventing IBD in a subject having an IDO2 variant nucleic acid molecule. Any of the IBD therapeutic agents that treat, prevent, or inhibit IBD described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

The present disclosure also provides IDO2 agonists for use in the treatment or prevention of IBD in a subject that is heterozygous or homozygous for an IDO2 variant nucleic acid molecule. Any of the IDO2 agonists described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

The present disclosure also provides IDO2 agonists in the preparation of a medicament for treating or preventing IBD in a subject that is heterozygous or homozygous for an IDO2 variant nucleic acid molecule. Any of the IDO2 agonists described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39989741:TG:T (Ala191fs), 8:39989787:C:T, or 8:39989793:A:T, or is an mRNA molecule produced therefrom, or is a cDNA molecule produced from the mRNA molecule.

The present disclosure also provides IBD therapeutic agents that treat, prevent, or inhibit IBD for use in the treatment or prevention of IBD in a subject having a SMAD2 variant nucleic acid molecule. Any of the IBD therapeutic agents that treat, prevent, or inhibit IBD described herein can be used herein. Any of the SMAD2 variant nucleic acid molecules disclosed herein can be used herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

The present disclosure also provides uses of IBD therapeutic agents that treat, prevent, or inhibit IBD for use in the preparation of a medicament for treating or preventing IBD in a subject having a SMAD2 variant nucleic acid molecule. Any of the IBD therapeutic agents that treat, prevent, or inhibit IBD described herein can be used herein. Any of the SMAD2 variant nucleic acid molecules disclosed herein can be used herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

The present disclosure also provides SMAD2 agonists for use in the treatment or prevention of IBD in a subject that is heterozygous or homozygous for a SMAD2 variant nucleic acid molecule. Any of the SMAD2 agonists described herein can be used herein. Any of the SMAD2 variant nucleic acid molecules disclosed herein can be used herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

The present disclosure also provides SMAD2 agonists in the preparation of a medicament for treating or preventing IBD in a subject that is heterozygous or homozygous for a SMAD2 variant nucleic acid molecule. Any of the SMAD2 agonists described herein can be used herein. Any of the SMAD2 variant nucleic acid molecules disclosed herein can be used herein. In some embodiments, the SMAD2 variant nucleic acid molecule is a SMAD2 variant genomic nucleic acid molecule that comprises the genetic variation 18:47869435:AC:A (Arg109SerfsTer54), 18:47845485:C:A, or 18:47896588:G:A, or is an mRNA molecule produced therefrom, or is a cDNA molecule produced from the mRNA molecule.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### Examples

### Example 1: General Methods

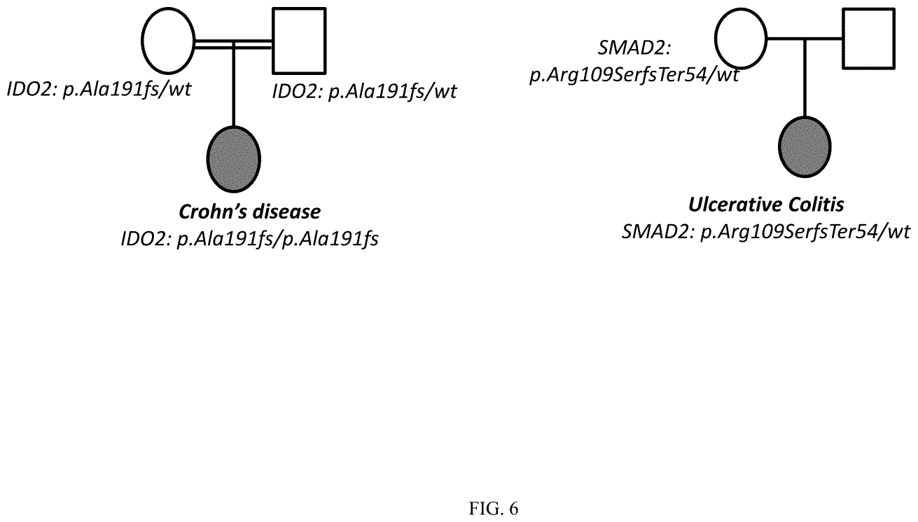

Diagnostic-like type of genetic analysis was performed in two unrelated families where the proband was diagnosed with early onset/infantile IBD (see, FIG. 6). The families were recruited as part of the The Boston Children's Hospital (BCH) Very Early Onset IBD Initiative cohort. Exome sequencing was carried out at the Regeneron Genetics Center (RGC) utilizing a customized automated sample preparation method. The targeted genome regions were captured using IDT xGen v1 and the captured libraries were sequenced on Illumina NovaSeq device. RGC DNAseq analytic pipeline was used to analyze the paired-end reads and identify genetic variations.

Variants were identified and annotated using the Ensembl, version 85 gene definitions and the GRCh38 human genome reference sequence. Pathogenic/likely pathogenic candidate variants in each proband were detected by utilizing an internal diagnostic-like analysis pipeline. To eliminate false positive variations, quality control filtering was used based on technical parameters such as minimum and maximum depth of coverage, base quality, mapping quality, and allelic imbalance. All variants were annotated using a variety of data resources including predicted functional effect at the transcript and protein level, allele/genotype frequency in multiple population genetic datasets, putative pathogenicity, conservation/constraint and known phenotypic associations. Variants were further evaluated using pedigree information, biological relevance and The American College of Medical Genetics and Genomics (ACMG) guidelines.

Exome-wide gene-based meta-analyses were performed using ten study cohorts totaling 28,779 IBD cases and 1,012,810 controls, including data from the UK Biobank (UKB), Geisinger Health System (GHS), Mount Sinai (Sinai) and the Mayo-clinic. These analyses estimated associations of sets of rare nonsyn(onymous variants, aggregated for each gene based on predicted variant function, with the risk of having Crohn's disease, ulcerative colitis, or either condition. Crohn's disease and ulcerative colitis were defined as the presence of ICD10 codes K50 and K51, respectively, in the electronic health records available on cohort participants. Statistical analyses were carried out with Firth's Bias-Reduced logistic regression implemented in Regenie v3.2.9. This approach produced association statistics for each aggregated variant set by gene, for each of the three conditions.

### Example 2: Identification of IBD-Causing Mutations in IDO2 and SMAD2

Diagnostic-like analysis performed in two families where the proband was diagnosed with early onset/infantile IBD identified 2 candidate likely pathogenic variants that can potentially explain the IBD phenotype in each proband (see, FIG. 6). In one of the probands, homozygous inherited from both parents frameshift variant 8:39989741:TG:T (p. Ala191fs) in the IDO2 gene was identified. In another proband, inherited heterozygous frameshift 18:47869435:AC:A (p. Arg109SerfsTer54) in the SMAD2 gene was identified. Both variants were classified as likely pathogenic according to ACMG criteria. IDO2 currently is not associated with any human disease, therefore the phenotype of 9 other homozygous carriers of different predicted loss of function variants (pLoFs) (8:39989787:C:T, 8:39989793:A:T, 8:39989793:A:T, 8:39989793:A:T) in IDO2 was investigated in detail in an internal RGC database. The majority of these homozygous carriers were found to have immune-related and gastrointestinal phenotypes e.g., gastritis and duodenitis, noninfective gastroenteritis and colitis, rheumatoid arthritis, diseases of esophagus, irritable bowel syndrome without diarrhea, other functional intestinal disorders, nausea and vomiting, other diseases of intestine, allergic asthma and chronic bronchitis.

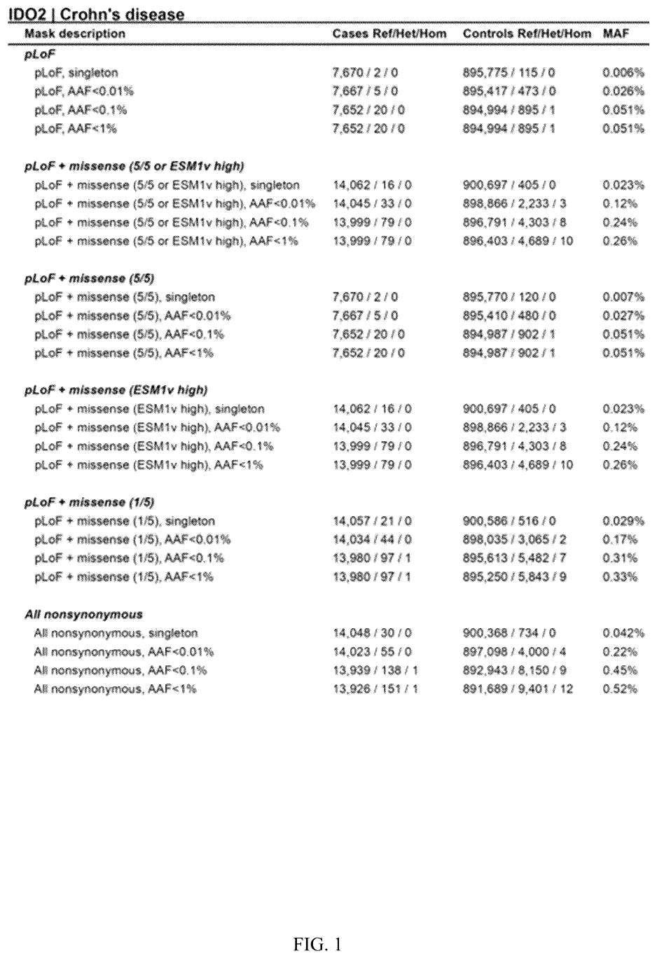

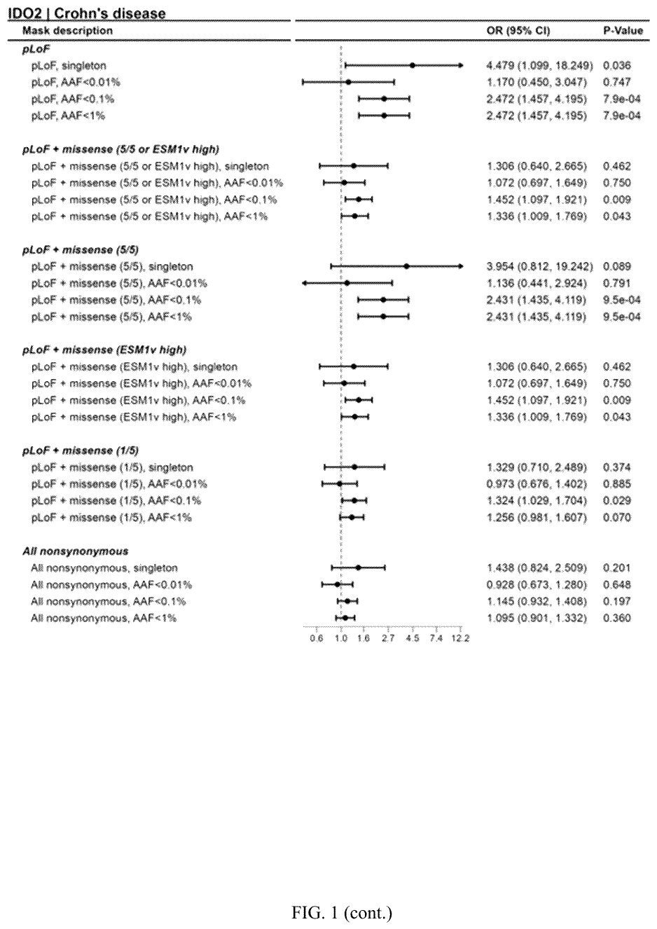

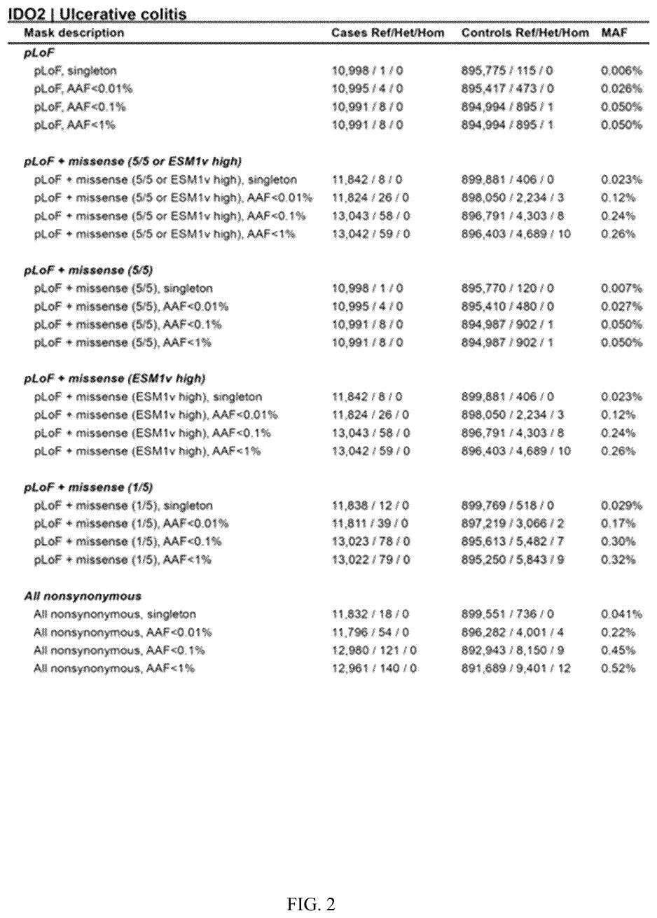

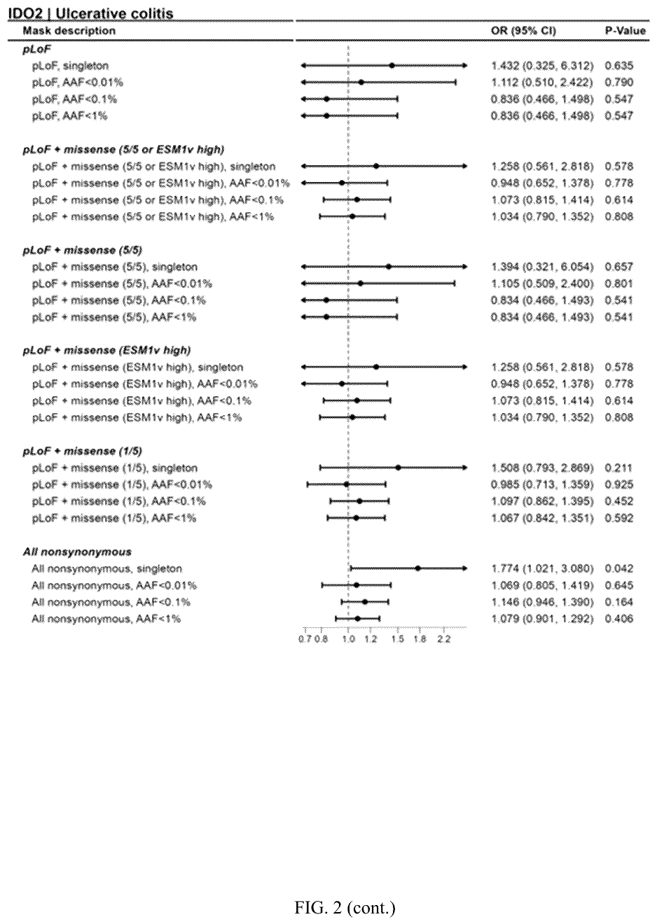

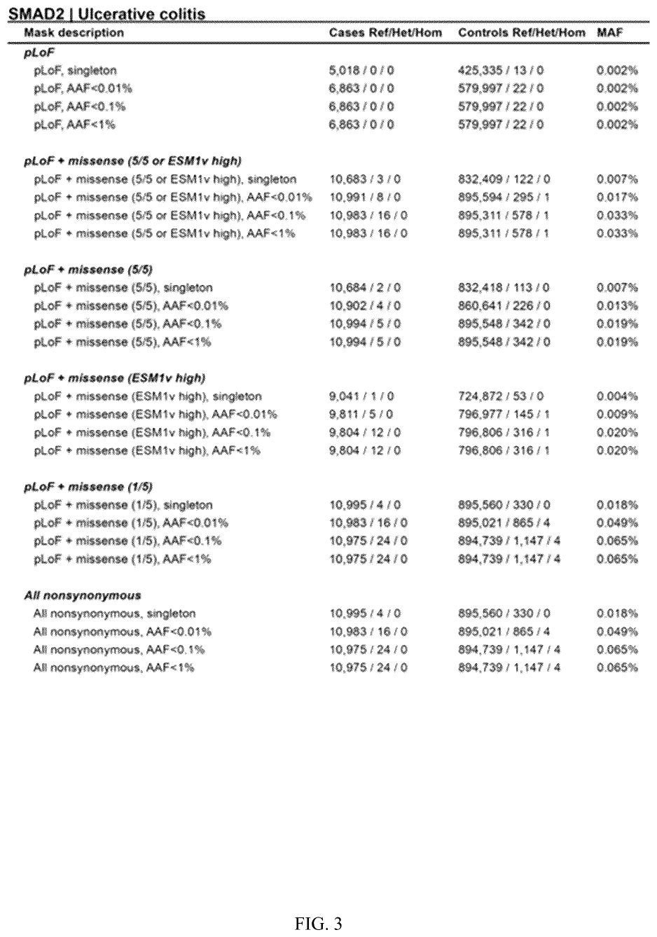

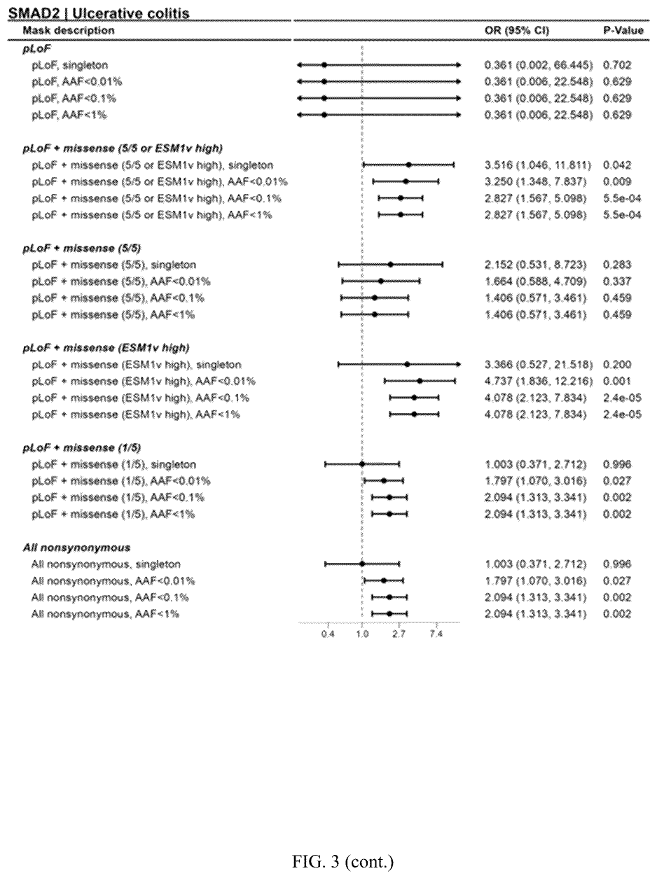

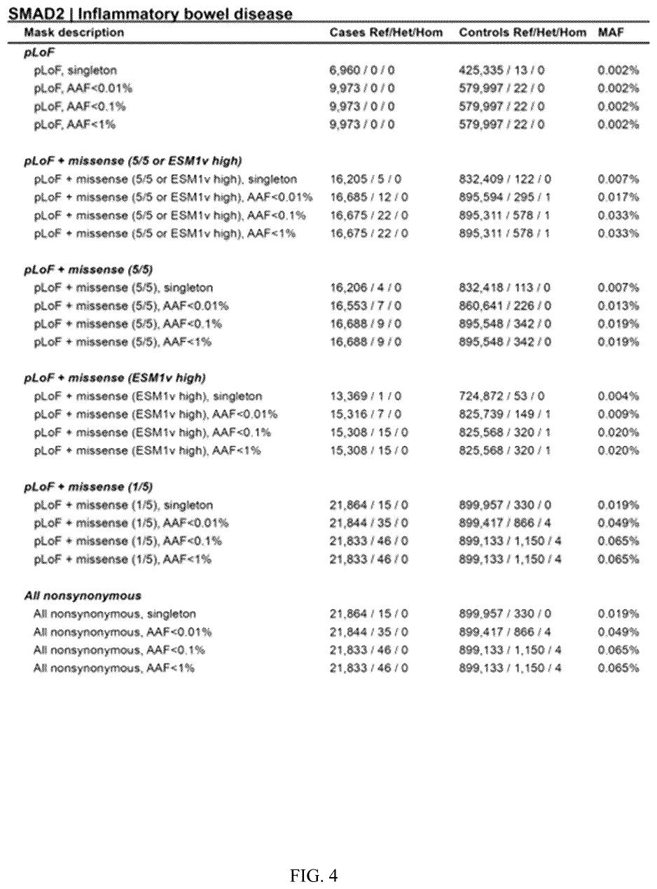

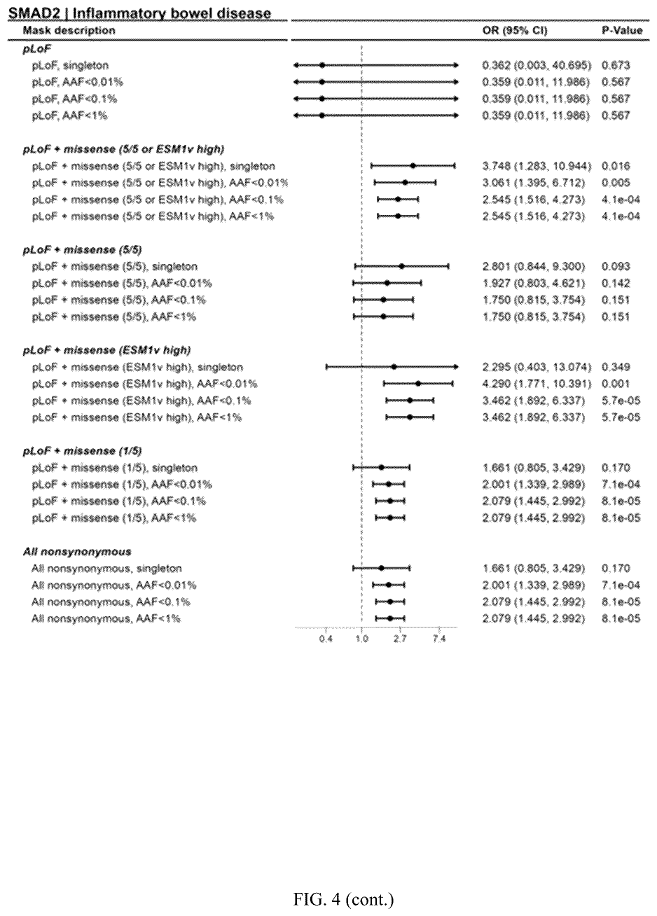

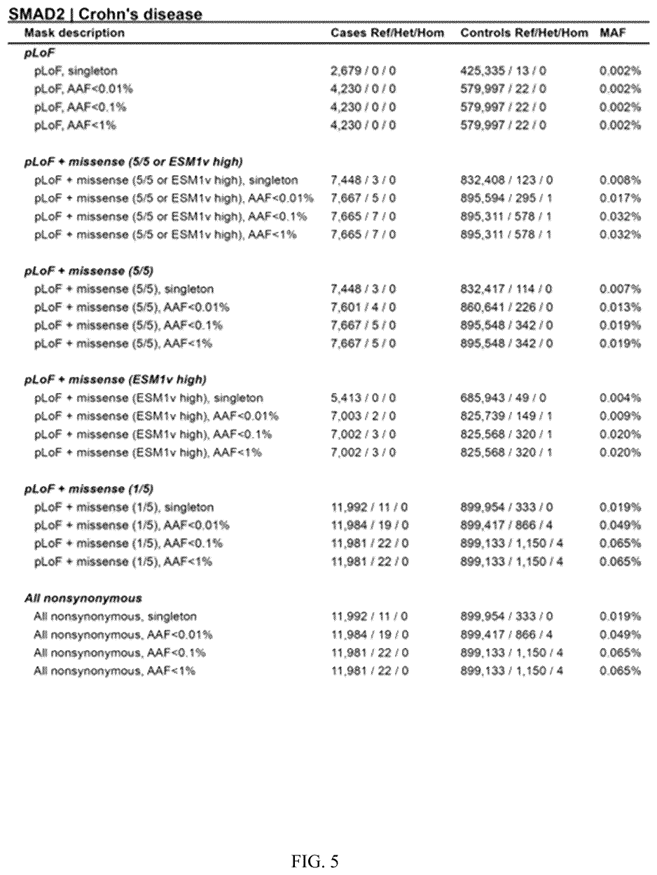

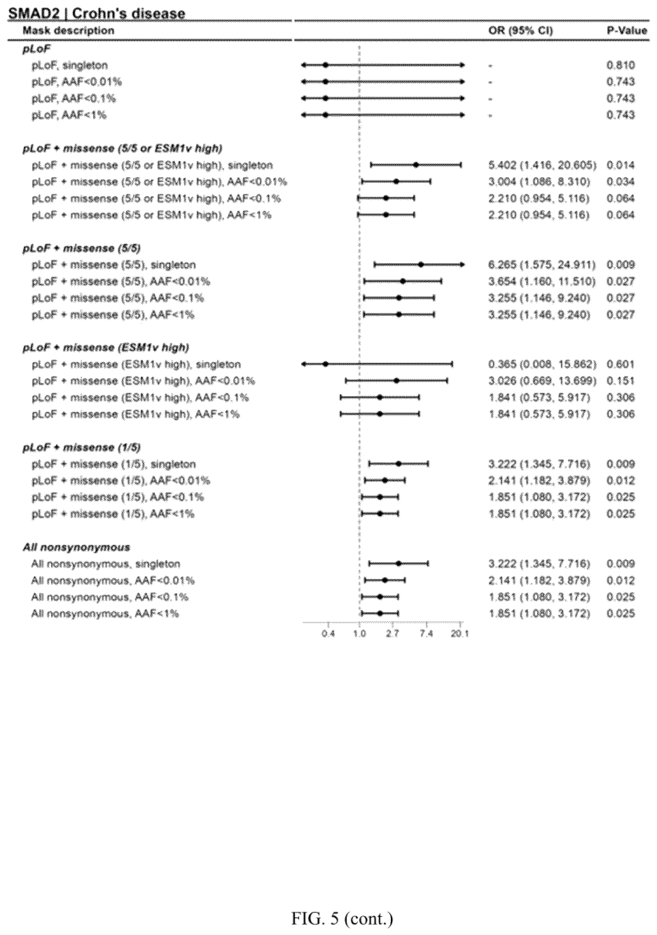

In order to confirm these findings of the diagnostic like analysis gene-based rare-variant burden tests aggregating effects of deleterious variant sets were performed within IDO2 and SMAD2 by predicted function and allele frequency (see, FIGS. 1-5). These tests identified associations between rare (alternative allele frequency (AAF)<1%) variants predicted to cause loss of function in IDO2 and a 2.47-fold increase in Crohn's disease risk (p=7.9×10⁻⁴), and between rare highly-deleterious variants in SMAD2 with a 4.1-fold increase in ulcerative colitis (p=2.4×10⁻⁵).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having Inflammatory Bowel Disease (IBD) or at risk of developing IBD, the method comprising administering an Indoleamine 2,3-Dioxygenase 2 (IDO2) agonist to the subject.

2. The method of claim 1, wherein the subject is heterozygous or homozygous for an IDO2 variant nucleic acid molecule.

3. The method of claim wherein the subject is also administered an IBD therapeutic agent.

4. The method of claim 1, further comprising detecting the presence or absence of an IDO2 variant nucleic acid molecule in a biological sample from the subject.

5. The method of claim 4, further comprising administering an IBD therapeutic agent in an amount that is the same as a standard dosage amount to the subject when the IDO2 variant nucleic acid molecule is absent from the biological sample.

6. The method of claim 4, further comprising administering an IBD therapeutic agent in an amount that is the same as or less than a standard dosage amount to the subject when the subject is heterozygous or homozygous for the IDO2 variant nucleic acid molecule.

7. The method of claim 4, wherein the IDO2variant nucleic acid molecule comprises a splice-site variant, a stop-gain variant, a start-loss variant, a stop-loss variant, a frameshift variant, a missense variant, an in-frame indel variant, and/or a variant that encodes a truncated IDO2 variant polypeptide.

8. The method of 7claim 4, wherein the IDO2 variant nucleic acid molecule comprises the genetic variation 8:39989741:TG:T (Ala191fs), 8:39989787:C:T, or 8:39989793:A:T.

9. A method of treating a subject having Inflammatory Bowel Disease (IBD) or at risk of developing IBD by administering an IBD therapeutic agent, the method comprising:
determining or having determined whether the subject has an Indoleamine 2,3-Dioxygenase 2 (IDO2) variant nucleic acid molecule, by:
obtaining or having obtained a biological sample from the subject; and
performing or having performed a sequence analysis on the biological sample to determine if the subject has a genotype comprising an IDO2 variant nucleic acid molecule; and
administering or continuing to administer the IBD therapeutic agent in an amount that is the same as a standard dosage amount to a subject that is IDO2 reference; or
administering or continuing to administer the IBD therapeutic agent in an amount that is the same as or less than a standard dosage amount, and/or an IDO2 agonist to a subject that is heterozygous or homozygous for the IDO2 variant nucleic acid molecule;
wherein the presence of the IDO2 variant nucleic acid molecule indicates the subject has an increased risk of developing IBD.

10-11. (canceled)

12. The method of claim 9, wherein the IDO2 variant nucleic acid molecule comprises a splice-site variant, a stop-gain variant, a start-loss variant, a stop-loss variant, a frameshift variant, a missense variant, an in-frame indel variant, and/or a variant that encodes a truncated IDO2 variant polypeptide.

13. The method of claim 9, wherein the IDO2 variant nucleic acid molecule comprises the genetic variation 8:39989741:TG:T (Ala191fs), 8:39989787:C:T, or 8:39989793:A:T.

14. A method of identifying a subject having an increased risk of developing Inflammatory Bowel Disease (IBD), the method comprising:
determining or having determined the presence or absence of an Indoleamine 2,3-Dioxygenase 2 (IDO2) variant nucleic acid molecule in a biological sample obtained from the subject;
wherein:
when the subject is IDO2 reference, then the subject has a decreased risk of developing IBD; and
when the subject is heterozygous or homozygous for the IDO2 variant nucleic acid molecule, then the subject has an increased risk of developing IBD.

15-25. (canceled)

26. A method of treating a subject having Inflammatory Bowel Disease (IBD) or at risk of developing IBD, the method comprising administering a SMAD Family Member 2 (SMAD2) agonist to the subject.

27. The method of claim 26, wherein the subject is heterozygous or homozygous for a SMAD2 variant nucleic acid molecule.

28. The method of claim 26, wherein the subject is also administered an IBD therapeutic agent.

29. The method of claim 26, further comprising detecting the presence or absence of a SMAD2 variant nucleic acid molecule in a biological sample from the subject.

30. The method of claim 29, further comprising administering an IBD therapeutic agent in an amount that is the same as a standard dosage amount to the subject when the SMAD2 variant nucleic acid molecule is absent from the biological sample.

31. The method of claim 29, further comprising administering an IBD therapeutic agent in an amount that is the same as or less than a standard dosage amount to the subject when the subject is heterozygous or homozygous for the SMAD2 variant nucleic acid molecule.

32. The method of claim 29, wherein the SMAD2 variant nucleic acid molecule comprises a splice-site variant, a stop-gain variant, a start-loss variant, a stop-loss variant, a frameshift variant, a missense variant, an in-frame indel variant, and/or a variant that encodes a truncated SMAD2 variant polypeptide.

33. The method of claim 29, wherein the SMAD2 variant nucleic acid molecule comprises the genetic variation 18:47869435:AC:A (Arg109SerfsTer54), 18:47845485:C:A, or 18:47896588:G:A.

34. A method of treating a subject having Inflammatory Bowel Disease (IBD) or at risk of developing IBD by administering an IBD therapeutic agent, the method comprising:
determining or having determined whether the subject has a SMAD Family Member 2 (SMAD2) variant nucleic acid molecule, by:
obtaining or having obtained a biological sample from the subject; and
performing or having performed a sequence analysis on the biological sample to determine if the subject has a genotype comprising a SMAD2 variant nucleic acid molecule; and
administering or continuing to administer the IBD therapeutic agent in an amount that is the same as a standard dosage amount to a subject that is SMAD2 reference; or
administering or continuing to administer the IBD therapeutic agent in an amount that is the same as or less than a standard dosage amount, and/or a SMAD2 agonist to a subject that is heterozygous or homozygous for the SMAD2 variant nucleic acid molecule;
wherein the presence of the SMAD2 variant nucleic acid molecule indicates the subject has an increased risk of developing IBD.

35-36. (canceled)

37. The method of claim 34, wherein the SMAD2 variant nucleic acid molecule comprises a splice-site variant, a stop-gain variant, a start-loss variant, a stop-loss variant, a frameshift variant, a missense variant, an in-frame indel variant, and/or a variant that encodes a truncated SMAD2 variant polypeptide.

38. The method of claim 34, wherein the SMAD2 variant nucleic acid molecule comprises the genetic variation 18:47869435:AC:A (Arg109SerfsTer54), 18:47845485:C:A, or 18:47896588:G:A.

39. A method of identifying a subject having an increased risk of developing Inflammatory Bowel Disease (IBD), the method comprising:
determining or having determined the presence or absence of a SMAD Family Member 2 (SMAD2) variant nucleic acid molecule in a biological sample obtained from the subject;
wherein:
when the subject is SMAD2 reference, then the subject has a decreased risk of developing IBD; and
when the subject is heterozygous or homozygous for the SMAD2 variant nucleic acid molecule, then the subject has an increased risk of developing IBD.

40-50. (canceled)
